(19) United States
(12) Patent Application Publication
(10) Pub. No.: US 2017/0158608 A1
(43) Pub. Date: Jun. 8, 2017

(54) Process for the Preparation of 2-Alkoxymethyl-1,4-Benzenediamines

(71) Applicant: The Procter & Gamble Company, Cincinnati, OH (US)

(72) Inventors: Bryan Patrick MURPHY, Loveland, OH (US); Garry Steven GARRETT, West Chester, OH (US); John Michael GARDLIK, Lebanon, OH (US)

(21) Appl. No.: 15/260,524

(22) Filed: Sep. 9, 2016

Related U.S. Application Data
(60) Provisional application No. 62/262,533, filed on Dec. 3, 2015.

(51) Int. Cl. C07C 213/10; C07C 209/74; A61K 8/41; C07C 213/08; C07D 207/404; A61Q 5/10; C07C 209/68; C07C 213/00

(52) U.S. Cl. CPC C07C 213/10; C07C 209/68; C07C 209/74; C07C 213/00; C07C 213/08; C07D 207/404; A61Q 5/10; A61K 8/411

(57) ABSTRACT

A process for synthesizing 2-methoxymethyl-1,4-benzenediamine (V-a), its derivatives of formula (V) and the salts thereof, which comprises a radical halogenation step of formula (II). The final product can be 2-methoxymethyl-1,4-benzenediamine of formula (IV-a).

### FIELD OF THE INVENTION

A process to synthesize 2-alkoxymethyl-1,4-benzenediamines (V), its derivatives, and salts thereof, comprising the protection of sensitive amine functionalities, radical halogenation mediated by halogenating agents, and alkoxylation. These compounds may be used as couplers and/or primary intermediates in compositions for dyeing keratin fibers.

### BACKGROUND OF THE INVENTION

CA 2,576,189 discloses the application of combinations of 2-methoxymethyl-1,4-benzenediamine (V-a) with various couplers and primary intermediates in oxidative dyeing compositions. U.S. Pat. No. 2,273,564 discloses a process to synthesize substituted 1,4-benzenediamine compounds with a substituent on the 2 position. U.S. Pat. No. 6,648,923 B1 discloses a process to synthesize 2-methoxymethyl-1,4-benzenediamine and the salts thereof.

The previous syntheses described above to reach 2-alkoxymethyl-1,4-benzenediamines, their derivatives, and salts thereof are not completely satisfactory.

Therefore, there is a need for a simple, industrially applicable, efficient, not expensive and high yield process to synthesize 2-alkoxymethyl-1,4-benzenediamines, their derivatives of formula (V), and salts thereof.

The key step of the process of this invention is a radical halogenation of the N-protected- or the N,N′diprotected-2-methyl-1,4-benzenediamine derivatives of formula (II) as key intermediates.

### SUMMARY OF THE INVENTION

A new process has now been developed to synthesize 2-alkoxymethyl-1,4-benzenediamines (V), its derivatives, and salts thereof, which permits a production of said compounds in a novel, high yield, cost effective and simple way.

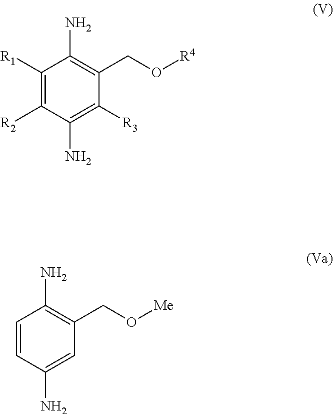

Said process comprises the a key step of performing a radical halogenation of compounds of formula (II) in the presence of a halogenation reagent, one or more radical initiators and/or heat, and one or more solvents to prepare compounds of formula (III):

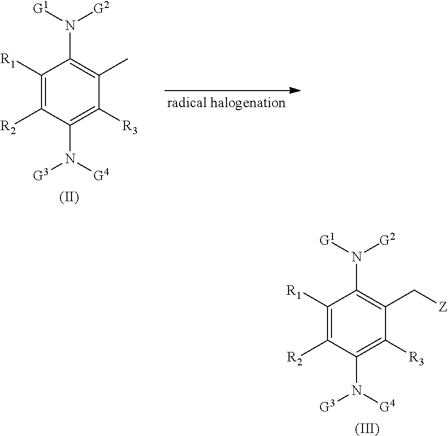

wherein R¹, R², R³ are substituents independently selected from the group consisting of
(a) C-linked substituents selected from the group consisting of:
   (i) mono- or poly-substituted or unsubstituted, straight or branched, aliphatic, heteroaliphatic, in particular alkyl, or heteroalkyl, mono- or poly-unsaturated aliphatic, in particular alkyl, or hetero unsaturated alkyl systems,
   (ii) mono- or poly-substituted or unsubstituted, mono- or poly-unsaturated aryl systems, and
   (iii) mono- or poly-substituted or unsubstituted, mono- or poly-unsaturated heteroaryl systems, and
   wherein said systems of (i), (ii) and (iii) comprise from about 1 to 10 carbon atoms and from about 0 to 5 heteroatoms selected from the group consisting of O, F, N, P and Si;
(b) S-linked substituents selected from the group consisting of SA¹, SO₂A¹, SO₃A¹, SSA¹, SOA¹, SO₂NA¹A², SNA¹A², and SONA¹A²;
(c) O-linked substituents selected from the group consisting of OA¹, ONA¹A²;
(d) N-linked substituents selected from the group consisting of NA¹A²; (NA¹A²A³)⁺, NA¹SA², NO₂; NA¹A²;
(e) substituents selected from the group consisting of COOA¹, CONA¹, CONA¹COA², C(═NA¹)NA¹A², CN, and X;
(f) fluoroalkyl substituents selected from the group consisting of mono-, poly-, and perfluoroalkyl systems comprising from 1 to 12 carbon atoms and from 0 to 4 heteroatoms; and
(g) H;

and mixtures thereof;

wherein A¹, A², and A³ are substituents independently selected from the group consisting of H; substituted or unsubstituted, straight or branched, alkyl, mono-or poly-unsaturated alkyl, heteroalkyl, aliphatic, heteroaliphatic, or heteroolefinic systems; substituted or unsubstituted, mono-or poly-cyclic aliphatic, aryl, or heterocyclic systems; and substituted or unsubstituted, mono-, poly-, or per-fluoro alkyl systems, or A^{l} and A² together with nitrogen atoms to which they are bound form a ring; wherein said systems comprise from 1 to 10 carbon atoms and from 0 to 5 heteroatoms selected from the group consisting of O, S, N, P, and Si; and wherein X is a halogen selected from the group consisting of F, Cl, Br, I, and mixtures thereof;

wherein G¹, G², G³ and G⁴ are substituents selected from the group consisting of hydrogen, oxygen, carbamates; 2,2,2-Trichloroethyl carbamate, 2-trimethylsilylethyl carbamate (Teoc), t-Butyl carbamate (BOC), allyl carbamate (Alloc), benzyl carbamate (Cbz), m-nitrophenyl carbamate, alternatively amides; formamides, trifluoroacetamides, sulfonamides, p-toluenesulfonyl (Ts), trifluoromethanesulfonyl, trimethylsilylethanesulfonamide (SES), tert-butylsulfonyl (Bus), alternatively cyclic imides; N-phthalimide, N-succinimide, N-dithiasuccinoyl, N-2,3-diphenylmaleoyl and mixtures thereof;

wherein Z is a halogen selected from the group consisting of Cl, Br, I;

Said process further comprising a key step of protecting the primary amine functions of compounds of formula (Ia, Ib or Ic) in the presence of a protecting agent and one or more solvents to prepare compounds of formula (II)

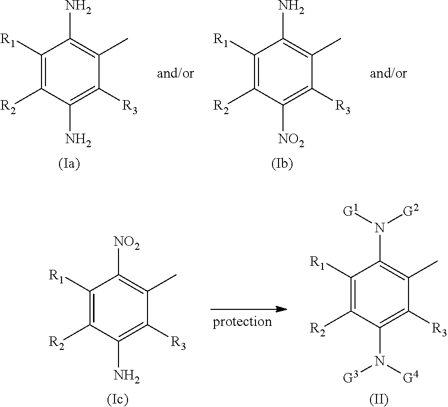

wherein R¹, R², R³ and G¹, G², G³, G⁴ are substituents as defined previously; and

the said process further comprises a step of alkoxylating compounds of formula (III) in the presence of an alkoxylating agent and one or more solvents to prepare compounds of formula (IV),

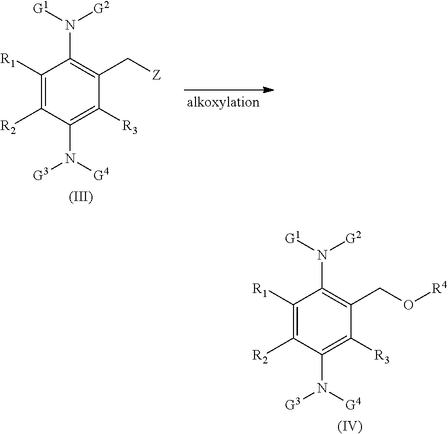

wherein R¹, R², R³ and G¹, G², G³, G⁴ are substituents as defined previously; and

wherein R⁴ is a substituent selected from the group consisting of a mono- or poly-substituted or unsubstituted, straight or branched, aliphatic, heteroaliphatic, in particular alkyl, or heteroalkyl, mono- or poly-unsaturated aliphatic, in particular alkyl, or hetero unsaturated alkyl systems and mixtures thereof; and

the said process may further comprise the steps of deprotecting compounds of formula (IV) in the presence of a deprotecting agent one or more solvents to prepare compounds of formula (Va), (Vb), and/or (Vc),

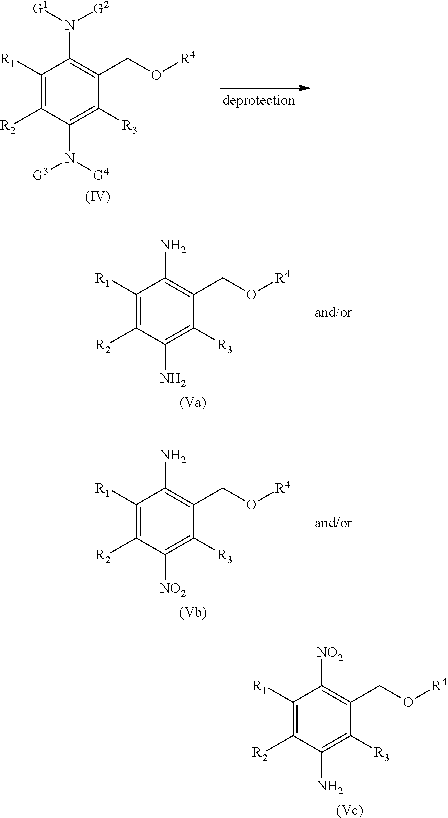

wherein R¹, R², R³, R⁴ and G¹, G², G³, G⁴ are substituents as defined previously.

Depending on the nature of substituents G¹, G², G³, G⁴, it may be necessary to carry out a hydrogenation or other type of reduction in order to prepare compounds of the formula of (Va),

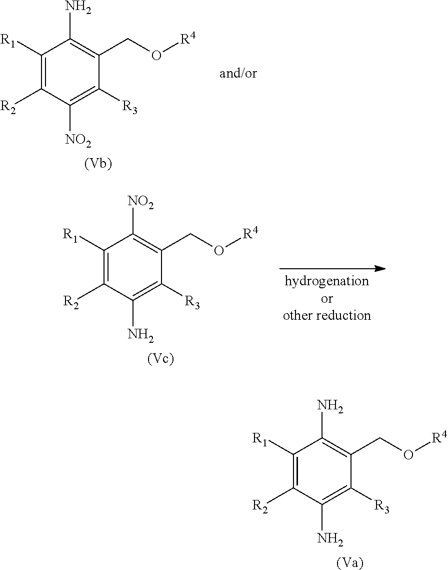

The said process may further comprise the step of transformation of compound (Va) into a salt of the formula (VI) in presence of an acid mHZ and one more solvents,

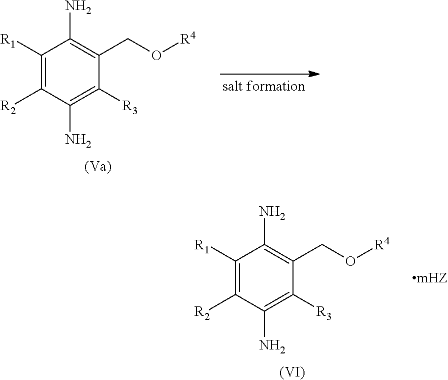

wherein R¹, R², R³ and R⁴ are substituents as defined previously.

### DETAILED DESCRIPTION OF THE INVENTION

Definitions:

In this specification, the term “substituted” refers to the following non limiting groups of; aliphatic, heteroaliphatic, in particular alkyl; or heteroalkyl; mono- or poly-unsaturated aliphatic, in particular alkyl; or hetero unsaturated alkyl systems; mono- or poly-unsaturated aryl systems; mono- or poly-unsaturated heteroaryl systems; and wherein said systems comprise from about 1 to 10 carbon atoms and from about 0 to 5 heteroatoms selected from the group consisting of O, F, N, P and Si and mixtures thereof.

In this specification, the term “aliphatic” refers to acyclic or cyclic, saturated or unsaturated carbon compounds, excluding aromatic compounds.

In this specification, the term “heteroaliphatic” refers to acyclic or cyclic compounds formally derived from aliphatic groups by replacement of one or more methine (—CH₃) and/or (—CH₂—) and/or (—CH—) groups by trivalent or divalent heteroatoms, respectively, in such a way as to maintain the continuous 6-electron system characteristic of aliphatic systems.

In this specification, the term “alkyl” refers to substituent derived from alkanes by removal of a hydrogen atom from any carbon atom: CₙH₂ₙ₋₁—. The groups derived by removal of a hydrogen atom from a terminal carbon atom of unbranched alkanes form a subclass of normal alkyl (n-alkyl) groups: H[CH₂]ₙ—. The groups RCH₂—, R₂CH— (R not equal to H), and R₃C— (R not equal to H) are primary, secondary and tertiary alkyl groups respectively.

In this specification, the term “heteroalkyl” refers to substituent derived from alkyl groups by replacement of one or more methyl (—CH₃) and/or methylene (—CH₂—) and/or methine (—CH—) groups by trivalent or divalent heteroatoms (N, O, S, P), respectively, in such a way as to maintain the continuous σ-electron system characteristic of alkyl systems.

In this specification, the term “aryl” refers to substituents derived from arenes by removal of a hydrogen atom from a ring carbon atom. The term “arene” has to be understood as monocyclic and polycyclic aromatic hydrocarbons.

In this specification, the term “heteroaryl” refers to the class of heterocyclic groups derived from heteroarenes by removal of a hydrogen atom from any ring atom; an alternative term is heteroaryl, e.g.

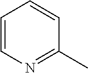

2-pyridyl (pyridin-2-yl),

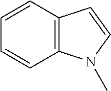

indol-1-yl. The term “heteroarene” refers to heterocyclic compounds formally derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2); an alternative term is heteroarenes. Comprehensive Heterocyclic Chemistry, Vol. 1, Ed. O.

Meth-Cohn, Pergamon, 1984, p. 3. E.g.

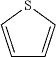

thiophene,

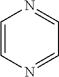

pyrazine.

In this specification, the abbreviation “Phth” refers to phthaloyl protecting group, i.e.

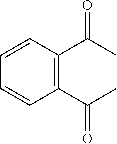

The different steps used in order to synthesize compounds of formula (V) will now be detailed.

I. The first step, step a) comprises protecting compounds (Ia), (Ib) and/or (Ic) in the presence of a protecting agent and optionally one or more solvents to prepare compounds of formula (II)

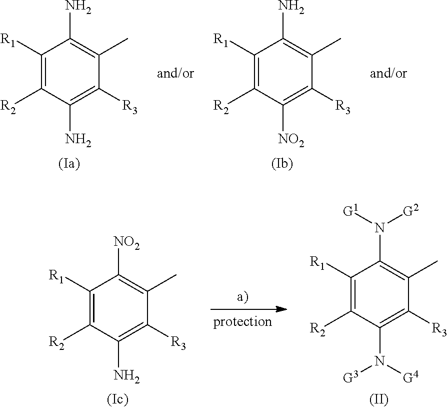

wherein R¹, R², R³, are substituents independently selected from the group consisting of
(a) C-linked substituents selected from the group consisting of:
   (i) mono- or poly-substituted or unsubstituted, straight or branched, aliphatic, heteroaliphatic, in particular alkyl, or heteroalkyl, mono- or poly-unsaturated aliphatic, in particular alkyl, or hetero unsaturated alkyl systems,
   (ii) mono- or poly-substituted or unsubstituted, mono- or poly-unsaturated aryl systems, and
   (iii) mono- or poly-substituted or unsubstituted, mono- or poly-unsaturated heteroaryl systems, and
   wherein said systems of (i), (ii) and (iii) comprise from about 1 to 10 carbon atoms and from about 0 to 5 heteroatoms selected from the group consisting of O, F, N, P and Si;
(b) S-linked substituents selected from the group consisting of SA¹, SO₂A¹, SO₃A¹, SSA¹, SOA¹, SO₂NA¹A², SNA¹A², and SONA¹A²;
(c) O-linked substituents selected from the group consisting of OA¹, ONA¹A²;
(d) N-linked substituents selected from the group consisting of NA¹A²; (NA¹A²A³)⁺, NA¹SA², NO₂; NA¹A²;
(e) substituents selected from the group consisting of COOA¹, CONA¹, CONA¹COA², C(═NA¹)NA¹A², CN, and X;
(f) fluoroalkyl substituents selected from the group consisting of mono-, poly-, and per-fluoro alkyl systems comprising from 1 to 12 carbon atoms and from 0 to 4 heteroatoms; and
(g) H;

and mixtures thereof;

wherein A¹, A², and A³ are substituents independently selected from the group consisting of H; substituted or unsubstituted, straight or branched, alkyl, mono-or poly-unsaturated alkyl, heteroalkyl, aliphatic, heteroaliphatic, or heteroolefinic systems; substituted or unsubstituted, mono-or poly-cyclic aliphatic, aryl, or heterocyclic systems; and substituted or unsubstituted, mono-, poly-, or per-fluoro alkyl systems, or A¹ and A² together with nitrogen atoms to which they are bound to form a ring; wherein said systems comprise from 1 to 10 carbon atoms and from 0 to 5 heteroatoms selected from the group consisting of O, S, N, P, and Si; and wherein X is a halogen selected from the group consisting of F, Cl, Br, I, and mixtures thereof;

wherein G¹, G², G³ and G⁴ are substituents selected from the group consisting of hydrogen, oxygen, carbamates; 2,2,2-Trichloroethyl carbamate, 2-trimethylsilylethyl carbamate (Teoc), t-Butyl carbamate (BOC), allyl carbamate (Alloc), benzyl carbamate (Cbz), m-nitrophenyl carbamate, alternatively amides; formamides, trifluoroacetamides, sulfonamides, p-toluenesulfonyl (Ts), trifluoromethanesulfonyl, trimethylsilylethanesulfonamide (SES), tert-butylsulfonyl (Bus), alternatively cyclic imides; N-phthalimide, N-succinimide, N-dithiasuccinoyl, N-2,3-diphenylmaleoyl and mixtures thereof.

Non limiting examples of solvents for the protecting step a) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof.

Non limiting examples of protecting agents for the protecting step a) can be selected from the group consisting of carbamates, 2,2,2-Trichloroethyl carbamate, 2-trimethylsilylethyl carbamate (Teoc), t-Butyl carbamate (BOC), allyl carbamate (Alloc), benzyl carbamate (Cbz), m-nitrophenyl carbamate; amides, acetamide, formamides, benzamides, trifluoroacetamides, sulfonamides, p-toluenesulfonyl (Ts), trifluoromethanesulfonyl, trimethylsilylethanesulfonamide (SES), tert-butylsulfonyl (Bus); cyclic imides, N-phthalimido, N-succinimido, N-4-nitrophthalimido, N-tetrachlorophthalimido, N-dithiasuccinimido, N-2,3-diphenylmaleimido.

II. The second, step b), radical halogenation, consists of inserting a halogen function to compound (II) in the presence of an halogen source, one or more catalysts and/or heat and one or more solvents to prepare compounds of formula (III):

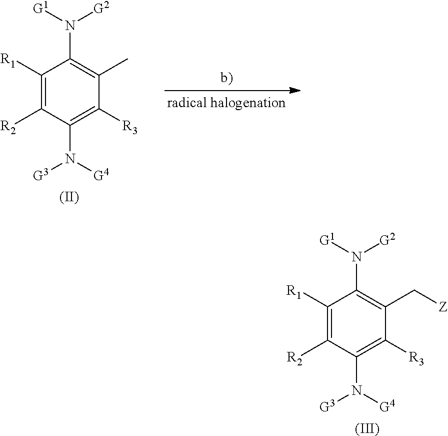

wherein R¹, R², R³, R⁴, G¹, G², G³, G⁴ are substituents as defined previously; and wherein Z is a halogen selected from the group consisting of Cl, Br, I;.

Non limiting examples of solvents for the radical halogenation step b) comprise water, pentane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof.

Non limiting examples of halogen source for the radical halogenation step b) comprise N-chlorosuccinimide, N-bromosuccinimide, N-iodosuccinimide, N-chlorophthalimide, N-bromophthalimide, N-iodophthalimide, N-chloroimidazole, N-bromoimidazole, N-chloroacetamide, N-bromoacetamide, N-iodoacetamide, N-chlorosaccharin, N-bromosaccharin, N-iodosaccharin, hydrochloric acid, hydrobromic acid, hydroiodic acid, sodium chlorate, sodium bromate, sodium iodoate, chlorodimethylsulfonium chloride, bromodimethylsulfonium bromide, dichloroisocyanuric acid, dibromoisocyanuric acid, diiodoisocyanuric acid, chlorine, bromine, iodine, carbon tetrachloride, carbon tetrabromide, carbon tetraiodide, and mixtures thereof.

Non limiting examples of radical initiators for the radical halogenation step b) comprise AIBN, ABCN, benzoyl peroxide, potassium persulfate, t-butyl hydroperoxide, lauroyl peroxide, peracetic acid, heat, and mixtures thereof.

III. The third step, step c), consists of performing an alkoxylation of compound (III) in the presence of an alkoxylating agent and in one or more solvents to prepare compounds of formula (IV):

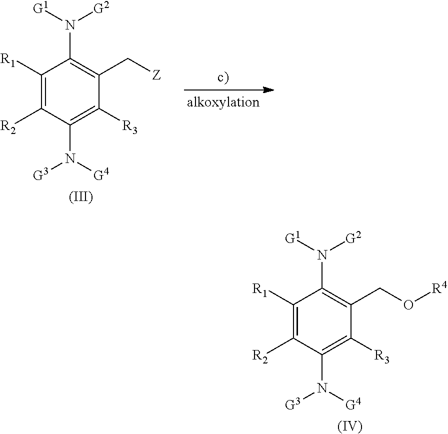

wherein R¹, R², R³, R⁴, G¹, G², G³, G⁴ are substituents as defined previously; and wherein Z is a substituent as defined previously.

Non limiting examples of solvents for the alkoxylation step c) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol, and mixtures thereof.

Non limiting examples of alkoxy source for the alkoxylation step c) comprise typical alcohols, sodium alkoxides, potassium alkoxides, zinc alkoxides, calcium alkoxides, magnesium alkoxides, tantalum alkoxides, tributyltin alkoxides, salts and mixtures thereof. For example, if methoxylation is desired for this alkoxyation step c), non-limiting examples of methoxy source comprise methanol, sodium methoxide, potassium methoxide, zinc methoxide, calcium methoxide, magnesium methoxide, tantalum methoxide, tributyltin methoxide, FeSO₄, salts and mixtures thereof.

IV The fourth step, step d), which may be done in the same reaction vessel as the third step if an activating group such as a nitro group is present, consists of performing a deprotection reaction by reacting compounds of formula (IV) in presence of a deprotecting agent and one or more solvents to prepare compounds of formula (Va), (Vb), and/or (Vc):

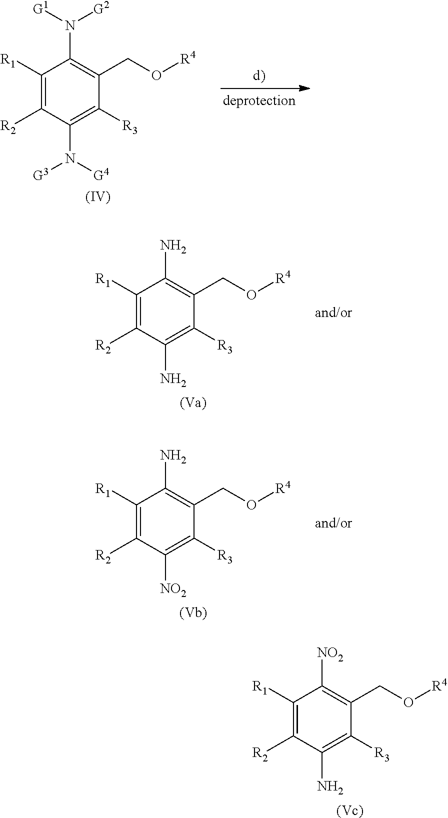

wherein R¹, R², R³, R⁴ and G¹, G², G³, G⁴ are substituents as defined above.

Non limiting examples of solvents for the deprotection step e) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof.

The skilled person would use his common general knowledge and the literature available to chose the right conditions to perform the deprotection step d).

Non limiting examples of deprotecting agent for the deprotection step d) comprise hydrazine, hydrazine acetate, phenylhydrazine, sodium sulphide, sodium borohydride, N-methylamine, ethylenediamine, N,N-dimethylpropylamine, hydroxylamine, methanesulfonic acid, hydroxides, alkoxides, and mixtures thereof.

V. A fifth step, step e), depending on the nature of substituents G¹, G², G³, G⁴, may be necessary to carry out a hydrogenation or other type of reduction in order to prepare compounds of the formula of (Va),

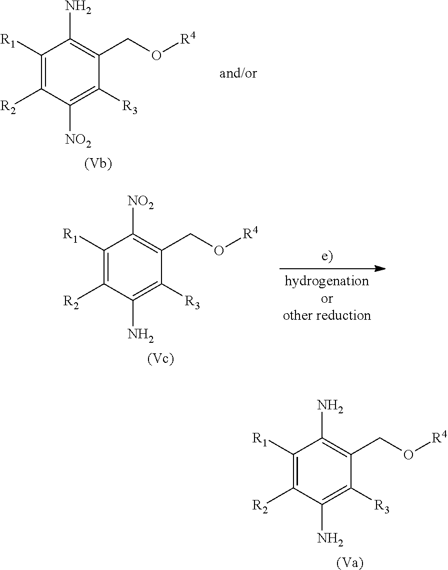

Non limiting examples of hydrogenation catalysts or other reducing agents comprise Raney nickel, nickel, palladium, Lindlar's catalyst, cobalt, copper chromite, platinum, platinum oxide, rhenium, tin(II) chloride, titanium(III) chloride, zinc, samarium, iron, and mixtures thereof.

VI. An optional step, step f), consists of transforming compound (Va) in presence of an acid mHZ and one or more solvents into a salt of the formula (VI):

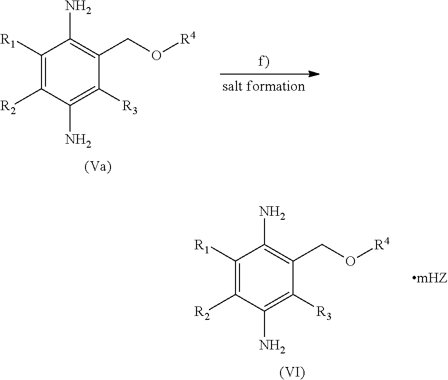

wherein R¹, R², R³, R⁴ are substituents as defined previously.

Non limiting examples of solvents for the salt formation step f) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof, in an embodiment ethanol and water.

Non limiting examples of acid mHZ for the salt formation step f) can be selected from the group consisting of D,L-malic acid, L-malic acid, D-malic acid, hydrochloric acid, hydrobromic acid, citric acid, acetic acid, lactic acid, succinic acid, tartaric acid, phosphoric acid, or sulfuric acid and mixtures thereof.

By HZ is meant any acid having an acid proton “H”. “Z” represents the rest of the molecule. For example if HZ═HCl, then Z═Cl. Another example can be if HZ═CH₃CO₂H, then Z═CH₃CO₂.

The value for “m” can be 0.5, 1, or 2, in an embodiment m=1.

The step f) can be performed at a temperature from −20° C. to 150° C., in an embodiment it is under inert gas atmosphere, at room temperature. By room temperature is meant 20-28° C.

VII. Application of the process above to the total synthesis 2-methoxymethyl-1,4-benzenediamine (Va) from 2-methyl-1,4-diaminotoluence (1a) as an illustrative example

The process described above can been utilized to synthesize 2-methoxymethyl-1,4-benzenediamine of formula (Va).

The steps are described in the following paragraphs.

VIIa. The first step, step a), consists of protecting 2-methyl-1,4-benzenediamine (Ia) in the presence of phthalic anhydride to prepare N,N-bis phthalimido-1,4-benzenediamine (IIa):

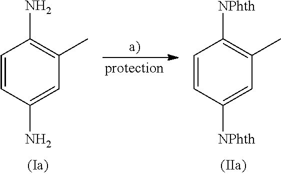

The protecting step a) is performed at a temperature from 0° C. to 286° C., in an embodiment 100-102° C.

The first step in the sequence to prepare 2-methoxymethyl-1,4-benzenediamine is the protection of 2-methyl-1,4-benzenediamine with phthalic anhydride to produce N, N-bis(phthalimido)-2-methyl-1,4-benzenediamine This can be accomplished inexpensively and in high yield according to a modification of the Schotten-Baumann method typically used for adding protecting groups to amino acids. For an explanation of this method see J March. March's Advanced Organic Chemistry (Wiley, 2001) (ISBN 0-471-58589-0). The modified conditions however, used base but no water.

VIIb. The second step, step b, consists of inserting a bromo-substituent by a radical bromination to N,N-bis-phthalimido-2-methyl-1,4-benzenediamine (IIa) in the presence of a non limiting examples source of bromine and one or more solvents to prepare N,N-bis phthalimido-bromomethyl-1,4-benzenediamine (IIa):

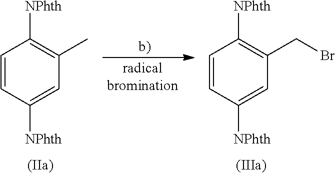

Non limiting examples of sources of bromine or the radical bromination step b) comprise N-bromosuccinimide, N-bromophthalimide, N-bromoimidazole, N-bromoacetamide, N-bromosaccharin, hydrobromic acid, sodium bromate, bromodimethylsulfonium bromide, dibromoisocyanuric acid, bromine, carbon tetrabromide, and mixtures thereof.

Non limiting examples of solvents for the radical bromination step b) comprise water, carbon tetrachloride, dichloromethane, acetonitrile, benzene, pyrrolidinone, pentane, cyclopentane, hexane, cyclohexane, toluene, 1,4-dioxane, chloroform, diethyl ether, dichloromethane, tetrahydrofuran, methyl-tetrahydrofuran, ethyl acetate, acetone, dimethylformamide, dimethyl sulfoxide, n-butanol, isopropanol, n-propanol, ethanol, methanol and mixtures thereof, more in an embodiment benzene and acetonitrile.

Non limiting examples of radical initiators for the radical bromination step b) comprise AIBN, ABCN, benzoyl peroxide, potassium persulfate, t-butyl hydroperoxide, lauroyl peroxide, peracetic acid, heat, and mixtures thereof.

The radical bromination step b) can be performed in heat at a temperature from about 30° C. to about 189° C., alternatively from about 40° C. to about 170° C., alternatively from about 50° C. to about 155° C., alternatively from about 70° C. to about 90° C., and alternatively from about 80° C. to about 82° C.

The second step in the sequence to prepare 2-methoxymethyl-1,4-benzenediamine is carried out according to the modification of standard methods for radical bromination. The standard conditions for radical benzylic (or allylic) bromination is the Wohl-Ziegler reaction. For a review of the Wohl-Ziegler reaction see C. Djerassi (1948). “Brominations with N-Bromosuccinimide and Related Compounds. The Wohl-Ziegler Reaction”. Chem. Rev. 43 (2): 271-317. A typical procedure involves the treatment of the benzyl compound in carbon tetrachloride, with N-bromosuccinimide and catalytic AIBN. Due to the extreme toxicity of carbon tetrachloride it can be necessary to use an alternative solvent(s). It is found that a mixture of benzene and acetonitrile can be a suitable for replacement of carbon tetrachloride. The reaction may also be promoted by heat to generate free radicals.

VIIc. The third step, step d), consists of performing a methoxylation by nucleophilic substitution of the bromo function on the benzylic position of compound III-a by a methoxy function in presence of methoxy source and one or more solvents to prepare N,N-bis phthalimido,2-methoxynethyl-benzen-1,4-diamine (IVa):

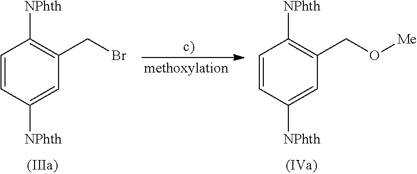

Non limiting examples of solvents for the methoxylation step c) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof, in an embodiment THF/MeOH.

Non limiting examples of methoxy source for the methoxylation step c) comprise methanol, sodium methoxide, potassium methoxide, zinc methoxide, calcium methoxide, magnesium methoxide, tantalum methoxide, tributyltin methoxide, salts and mixtures thereof. Optionally FeSO₄ can be employed with methanol to catalyze the methoxylation.

The methoxylation step c) can be performed at temperature from −20° C. to 150° C., in an embodiment 50° C. to 52° C.

Generally, this step is accomplished by heating N,N-bis-phthalimido-2-bromomethyl-1,4-benzenediamine with sodium methoxide in methanol. More recently it has been demonstrated by Girdhar Joshi & Subbarayappa Adimurthy (2011) (New Method for the Synthesis of Benzyl Alkyl Ethers Mediated by FeSO₄ , Synthetic Communications: An International Journal for Rapid Communication of Synthetic Organic Chemistry, 41:5, 720-728) that the action of easily recoverable FeSO₄ in the presence of methanol can be a mild, high yielding way to generate the 2-methoxymethyl side chain.

VIId. The fourth step, step d) consists of performing a deprotection by reacting N,N-bis-phthalimido-2-methoxymethyl-1,4-benzenediamine (IV-a) in presence of a deprotecting agent and one or more solvents to prepare 2-methoxymethyl-benzene-1,4-diamine (Va):

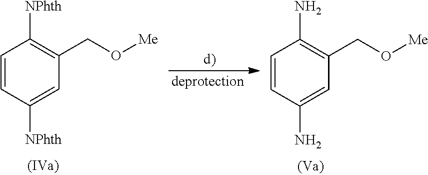

Non limiting examples of solvents for the deprotection step d) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof, in an embodiment ethanol.

Non limiting examples of deprotecting agent for the deprotection step d) comprise hydrazine, hydrazine acetate, hydrazine hydrate, phenylhydrazine, sodium sulphide, sodium borohydride, N-methylamine, ethylenediamine, N,N-dimethylpropylamine, hydroxylamine, methanesulfonic acid, in an embodiment hydrazine.

The deprotection step e) can be performed at temperature from −20° C. to 100° C., in an embodiment 20° C. to 28° C.

This step involves the deprotection of the protected amines (cleavage of the phthalimide protecting groups) in one of numerous ways as described in “Protective Groups in Organic Synthesis” by Peter G. M. Wuts, Theodora W. Greene, Wiley-Interscience; 4^{th} addition (Oct. 30, 2006) or “Protecting Groups” by Philip J. Kocienski, Thieme, Stuttgart; Auflage; 3^{rd} Revised edition (Feb. 14, 2005). Protecting groups are widely used in chemistry and the skilled person willing to use protecting groups during the process described in this invention would also judge on the conditions for the deprotection step during the process to remove them in order to reach compounds of formula Va. There are two primary means to accomplish this deprotection and these include the use of hydrazine to generate the phthalic hydrazide and/or the use of N-methylamine.

VIIe. An optional step, step f), consists of transforming compound (Va) in presence of an acid mHZ and one or more solvents into a salt of the formula (VI):

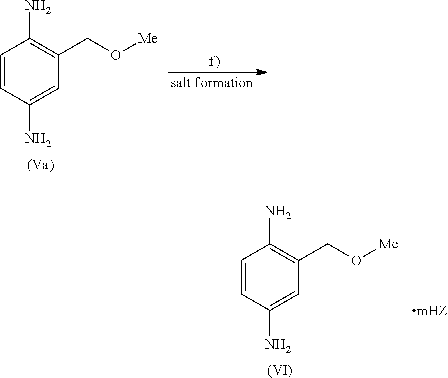

Non limiting examples of solvents for the salt formation step f) comprise pentane, cyclopentane, hexane, cyclohexane, benzene, toluene, 1,4-dioxane, chloroform, diethyl ether, dichloromethane, tetrahydrofuran, methyl-tetrahydrofuran, ethyl acetate, acetone, dimethylformamide, acetonitrile, dimethyl sulfoxide, n-butanol, isopropanol, n-propanol, ethanol, methanol and mixtures thereof, in an embodiment ethanol and water.

Non limiting examples of acid mHZ for the salt formation step can be selected from the group consisting of D,L-malic acid, L-malic, D-malic, hydrochloric, hydrobromic, citric, acetic, lactic, succinic, tartaric, phosphoric acid, or sulfuric acids and mixtures thereof.

By HZ is meant any acid having an acid proton “H”. “Z” represents the rest of the molecule. For example if HZ═HCl, then Z═Cl. Another example can be if HZ═CH₃CO₂H, then Z═CH₃CO₂.

The value for “m” can be 0.5, for 2, in an embodiment m=1.

This step can be performed at a temperature from −20° C. to 150° C., in an embodiment it is performed under inert gas atmosphere, at room temperature. By room temperature is meant 20-28° C.

### EXAMPLES

The present invention is further illustrated by the non-limiting examples that follow.

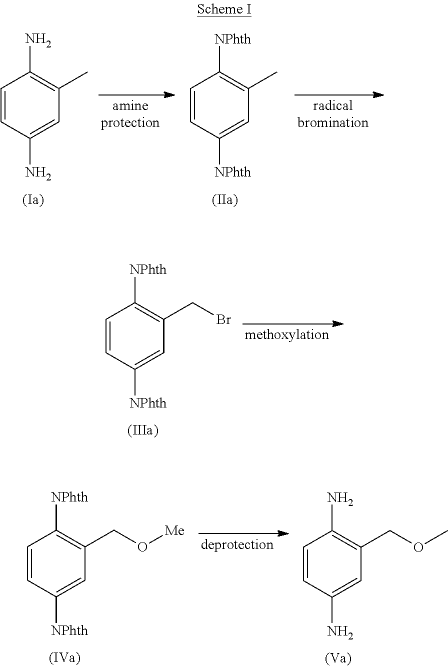

Experimental:

### 1. Synthesis of N,N-bis-phthalimido-2-methyl-1,4-benzenediamine (IIa)

2-methyl-1,4-benzenediamine (I-a) (10 g, 45.4 mmol) was dissolved in dioxane (400 mL). Phthalic anhydride (26.88 g, 181.8 mmol) and triethylamine (14.0 mL, 98.9 mmol) were added at room temperature. The resulting mixture was stirred overnight at reflux, after which the product had precipitated out of solution. The resulting reaction mixture was filtered while hot to collect the product. The filter cake was washed with water (2× 250 ml). The solids were transferred to a round bottom flask and azeotroped with acetone to remove water. The resulting solids were dried in vacuo to constant weight. N,N-bis-phthalimido-2-methyl-1,4-benzenediamine (IIa) (16.5 g, 95%) was obtained as a grey solid; ¹H-NMR (500 MHz, CDCl₃) δ 2.28 (s, 3H), 7.2-7.6 (m, 3H), 7.81 (d, 4H), 8.01 (d, 4H). IS-MS m/z MH⁺ 383 (100), M [H₂0-Na]⁺ 424 (90).

### 2. Synthesis of N,N-bis-phthalimido-2-bromomethyl-1,4-benzenediamine (IIIa)

N,N-bis-phthalimido-2-methyl-1,4-benzenediamine (II-a) (1.0 g, 2.62 mmol) was dissolved in a mixture of acetonitrile/benzene (1:1, 10 mL). N-bromosuccinimide (513 mg, 2.82 mmol) and AIBN (20 mg, 0.12 mmol) were added and the resulting mixture was heated to reflux for 24 h. Another portion of N-bromosuccinimide (513 mg) and AIBN (20 mg) were added with refluxing for and additional 24 h. The solvent was evaporated and the residue suspended in ethyl acetate. This mixture was stirred vigorously to sheer the brown solid. Filtration provided N,N-bis-phthalimido-2-bromomethyl-1,4-benzenediamine (IIIa) (880mg, 1.904 mmol, 73% yield) as a light gray solid. ¹H-NMR (500 MHz, (CD3)₂SO₂, δ 2.64 (s, 2H), 7.94-8.05 (m, 11H). IS-MS m/z MH³⁰ 459/461 (100), M [—Br]⁺ 381 (15)

### 3. Synthesis of N,N-bis phthalimido-2-methoxynethyl-1,4-benzenediamine (IVa)

N,N-bis-phthalimido-2-bromomethyl-1,4-benzenediamine (III-a) (700 mg, 1.51 mmol) was dissolved in MeOH/THF (1:1, 25 mL) followed by the addition of NaOMe (25%) (325 mg, 1.48 mmol). The resulting mixture was heated at 50° C. for 2-3 h. The methanol/THF was evaporated and the residue partitioned between dichloromethane (25 mL) and water (25 mL). The phases were separated and the solvent evaporated in vacuo to afford N,N-bis phthalimido-2-methoxynethyl-1,4-benzenediamine (IVa) (350mg, 56% yield) as pure solid; ¹NMR (500 MHz, CD₃Cl) δ 3.26 (s, 3H), 4.45 (s, 2H), 7.2-7.75 (m, 3H), 7.78-7.9 (d, 4H), 7.92-7.98 (d, 4H). IS-MS m/z M [—OCH₃]⁺ 381 (100), MH³⁰ 413 (65).

### 4. Synthesis of 2-methoxymethyl-1,4-benzenediamine (Va)

N,N-bis phthalimido-2-methoxymethyl-1,4-benzenediamine (IVa) (250 mg, 0.604 mmol) was dissolved in ethanol (5mL) and hydrazine (0.250 mL) added. The mixture was stirred at room temperature for 30 min. TLC analysis (50% ethyl acetate/hexane) indicated complete conversion to known product. The solvent was evaporated and the residue suspended in methanol. The insoluble solid was collected via filtration providing 2-methoxymethyl-1,4-benzenediamine (Va) (100mg, 0.604 mmol, quantitative yield) as a brown solid. ¹H-NMR (500 MHz, CDCl₃) δ 3.2-3.9 (m, 7H), 4.39 (s, 2H), 6.5 (s, 1H), 6.56 (s, 2H). IS-MS m/z M [—OCH₃]⁺ 122 (100), MH³⁰ 153 (10)

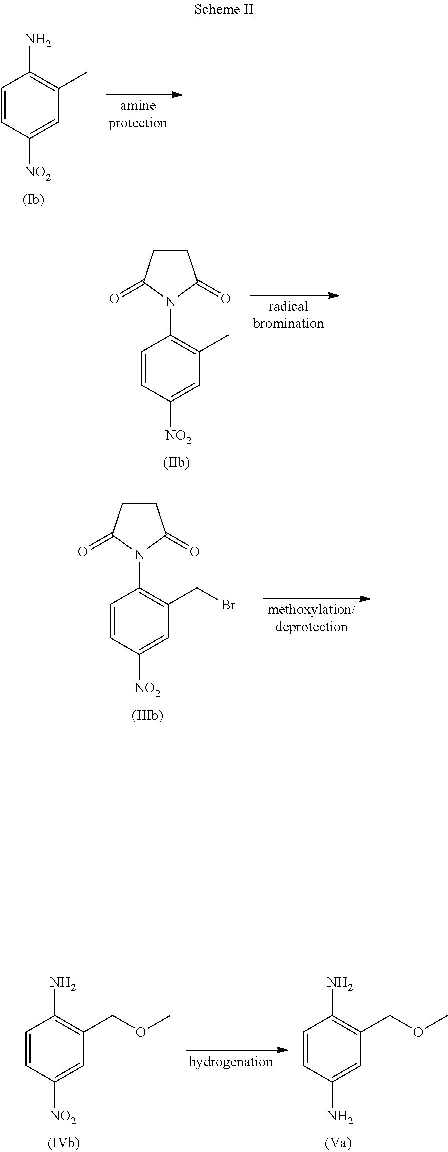 [SMILES: Cc1cc([N+](=O)[O-])ccc1N ; Cc1cc([N+](=O)[O-])ccc1N1C(=O)CCC1=O ; O=C1CCC(=O)N1c1ccc([N+](=O)[O-])cc1CBr ; COCc1cc([N+](=O)[O-])ccc1N ; COCc1cc(N)ccc1N]

### Experimental:

### 1. Synthesis of 1-(2-methyl-4-nitrophenyl)pyrrolidine-2,5-dione (IIb)

2-Methyl-4-nitroaniline (Ib) (5 g, 33 mmol) was dissolved in dioxane (200 mL). Succinic anhydride (7.2 g, 72 mmol) and triethylamine (10 mL, 72 mmol) were added at room temperature. The resulting mixture was stirred and refluxed for several days. The solvent was evaporated providing about 10 g of crude material. TLC analysis (50% EtOAc-hexanes) demonstrated complete consumption of starting material and the appearance of a new product at Rf=0.40. The solids were suspended in a mixture of ethyl acetate/dichloromethane and the soluble portion was added to 50 g of silica gel in a round bottom flask. The solvent was evaporated from the silica gel and this material was used to dry load a 230 g silica gel column. The material was eluted with ethyl acetate/hexanes gradient providing 1-(2-methyl-4-nitrophenyl)pyrrolidine-2,5-dione) (IIb), (1.0 g, 13%) as a white powder; ¹H-NMR (600 MHz, CDCl₃) δ 2.18 (s, 3H), 2.89 (m, 4H), 7.21 (s, 1H), 8.08 (dd, 1H), 8.13 (d, 1H). ¹³C-NMR 18.15, 28.74, 122.00, 126.11, 129.39, 136.88, 138.03, 148.06, 175.25 IS-MS MH⁺ m/z 235 (10), MH⁺ 194 (30), MH+ 153 (60), MH+ 135 (100)

### 2. Synthesis of 1-(2-bromomethyl-4-nitrophenyl)pyrrolidine-2,5-dione) (IIIb)

1-(2-Methyl-4-nitrophenyl)pyrrolidine-2,5-dione) (IIb) (0.5 g, 2.13 mmol) was dissolved in a mixture of acetonitrile/benzene (1:1, 10 mL). N-bromosuccinimide (455 mg, 2.56 mmol) and AIBN (35 mg, 0.21 mmol) were added and the resulting mixture was heated to reflux for 24 h. Two Additional portions of N-bromosuccinimide (455 mg) and AIBN (35 mg) were added at separate times with 16-20 hr refluxing in between. After the last period of reflux, TLC analysis (50% EtOAc/hexanes) demonstrated two closely eluting spots. UPLC analysis demonstrated good separation of these products. Chromatography on a C-18 RP-HPLC column separated the two materials providing 1-(2-bromomethyl-4-nitrophenyl)pyrrolidine-2,5-dione) (IIIb), (180 mg, 27%) as a white powder. ¹H-NMR (600 MHz, CDCl₃) δ 3.01 (m, 4H), 4.43 (s, 2H), 7.38 (d, 1H), 8.29 (dd, 1H) 8.38 (d, 1H). ¹³C-NMR 27.99, 28.76, 124.51, 126.02, 130.76, 136.89, 137.04, 147.92, 175.37

### 3. Synthesis of 2-(methoxymethyl)-4-nitroaniline (IVb)

1-(2-Bromomethyl-4-nitrophenyl)pyrrolidine-2,5-dione) (IIIb) (111 mg, 0.35 mmol) was dissolved in MeOH (10 mL). NaOMe (25%) (84 mg, 0.039 mmol) was added followed by heating to reflux for 30 minutes. TLC (50% EtOAc/hexanes) analysis demonstrated complete consumption of starting material. Evaporation provided 130 mg of a yellow semi-solid. Subsequent chromatography on silica gel provided 2-(methoxymethyl)-4-nitroaniline (IVb) 22 mg (35%). ¹H-NMR (600 MHz, CD₃Cl) δ 3.28 (s, 3H), 4.45 (s, 2H), 6.56 (d, 1H), 7.94 (d, 1H), 7.97 (dd, 4H). IS-MS m/z MH⁺ 183 (40), MH⁺ 166 (85), 150 (100).

### 4. Synthesis of 2-methoxymethyl-1,4-benzenediamine (Va)

Into a Parr hydrogenation bottle previously purged with argon, are placed a solution of 2-(methoxymethyl)-4-nitroaniline (5.0 g, 27.5 mmol) in ethyl acetate (50 mL) and 0.25 g of 10% palladium on carbon. The bottle is mounted on a Parr shaker and hydrogenation is carried out under 50-60 psig (64.7-74.7 psi or 446-515 kPa) of hydrogen pressure. The pressure is carefully monitored for hydrogen uptake and additional hydrogen is introduced to keep the pressure above 50 psig. The hydrogenation is carried out for 3.5 hours after which the catalyst is removed by vacuum filtration. The filtrate is concentrated to about 25 mL and toluene (25 mL) is added to precipitate the product, which is collected by vacuum filtration and dried at 60° C. under vacuum. The product yield is 4.5 g; ¹H-NMR (500 MHz, DMSO-d₆) δ 3.23 (s, 3H), 4.11 (s, 2H), 4.21 (s, 2H), 4.24 (s, 2H), 6.33 (dd, 1H), 6.37 (d, 1H), 6.41 (d, 1H).

Described herein is an embodiment 1 which is a process for preparing a primary intermediate for oxidative dyeing compositions, the process comprising performing a radical halogenation of one or more compounds of formula (II) in the presence of a halogenating agent, one or more radical initiators, heat and one or more solvents to prepare one or more compounds of formula (III):

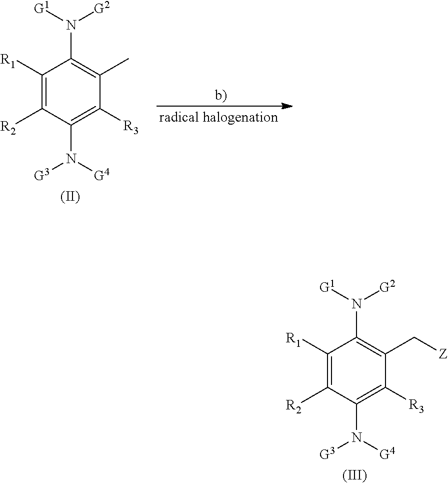

wherein R¹, R², and R³ are substituents independently selected from the group consisting of
   (a) C-linked substituents selected from the group consisting of:
      (i) aliphatic or heteroaliphatic substituents selected from the group consisting of mono- or poly-substituted or unsubstituted, straight or branched, cyclic or acyclic, saturated or unsaturated, and combinations thereof;
      (ii) aryl substituents selected from the group consisting of mono- or poly-substituted or unsubstituted, saturated or unsaturated, and combinations thereof;
      (iii) heteroaryl substituents selected from the group consisting of mono- or poly-substituted or unsubstituted, saturated or unsaturated, and combinations thereof;
      wherein said substituents of (i), (ii) and (iii) comprise from 1 to 10 carbon atoms and from 0 to 5 heteroatoms selected from the group consisting of O, F, N, P and Si;
   (b) S-linked substituents selected from the group consisting of SA¹, SO₂A¹, SO₃A¹, SSA¹, SOA¹, SO₂NA¹A², SNA¹A², and SONA¹A²;
   (c) O-linked substituents selected from the group consisting of OA¹ and ONA¹A²;
   (d) N-linked substituents selected from the group consisting of NA¹A²; (NA¹A²A³)⁺, NA¹SA², NO₂, and NA¹A²;
   (e) substituents selected from the group consisting of COOA¹, CONA¹, CONA¹COA², C(═NA¹)NA¹A², CN, and X;
   (f) fluoroalkyl substituents selected from the group consisting of mono-, poly-, and per-fluoro alkyl systems comprising from 1 to 12 carbon atoms and from 0 to 4 heteroatoms; and
   (g) H;
      and combinations thereof;
wherein A¹, A², and A³ are substituents independently selected from the group consisting of H, substituted or unsubstituted C1-C6 alkyl, substituted or unsubstituted C1-C6 heteroalkyl, substituted or unsubstituted aryl, substituted or unsubstituted C1-C6 heteroalkylaryl, substituted or unsubstituted heteroaryl, and/or substituted or unsubstituted cyclic heteroalkyl;
wherein G¹, G2, G³ and G⁴ are substituents selected from the group consisting of hydrogen, oxygen, carbamates, amides, sulfonamides, cyclic imides, and combinations thereof;
wherein G¹, G², G³ and G⁴ are not all oxygen; and
wherein Z is chlorine, bromine, or iodine.

In embodiment 2, the process according to embodiment 1 may further comprise the step of:
protecting amine functions in a compound of formula (I) in the presence of a protecting agent and one or more solvents to prepare the one or more compounds of formula (II)

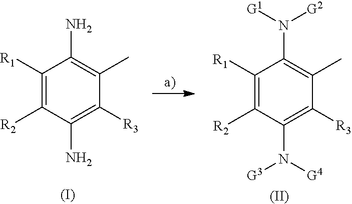

wherein R¹, R², R³ and G¹, G², G³, G⁴ are substituents as defined in embodiment 1.

In embodiment 3, the process according to embodiments 1 or 2 may further comprise the step of:
a) alkoxylating the one or more compounds of formula (III) in the presence of an alkoxylating agent and one or more solvents to prepare the one or more compounds of formula (IV),

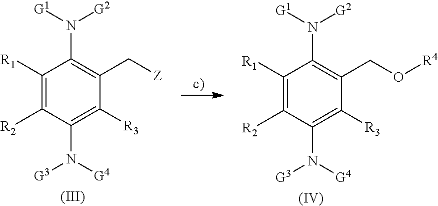

wherein R¹, R², R³, G¹, G², G³, G⁴ and Z are substituents as defined in embodiment 1; and
wherein R⁴ is a substituent independently selected from H, substituted or unsubstituted C1-C6 alkyl, substituted or unsubstituted C1-C6 heteroalkyl, substituted or unsubstituted aryl, substituted or unsubstituted C14-C6 heteroalkylaryl, substituted or unsubstituted heteroaryl, and/or substituted or unsubstituted heterocyclic aliphatic.

In embodiment 4, the process according to embodiment 3 may further comprise the step of:

a) deprotecting the one or more compounds of formula (IV) in the presence of a deprotecting agent and one or more solvents to prepare one or more compounds of formula (V):

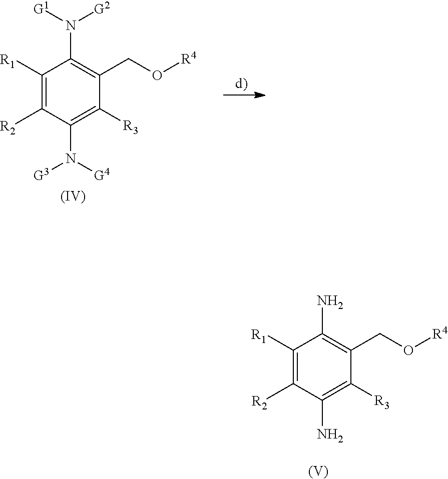

wherein R¹, R², R³, R⁴, G¹, G², G³, and G⁴ are substituents as defined in embodiment 3.

In embodiment 5, the process according to embodiment 4 may further comprise the step of transformation of the one or more compounds (V) into a salt of one or more compounds of formula (VI) in the presence of an acid mHZ and one more solvents:

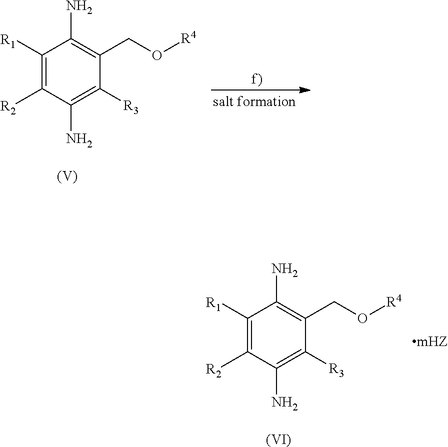

wherein R¹, R², R³ and R⁴ are substituents as defined in embodiment 4; and
wherein m is 0.5, 1, or 2; and
wherein HZ is selected from the group consisting of D,L-malic, L-malic, D-malic, hydrochloric, hydrobromic, phosphoric, citric, acetic, lactic, succinic, tartaric, sulfuric acids, and mixtures thereof.

Embodiment 6 is the process according to embodiments 4 or 5 wherein the amine protection step a), radical halogenation step b), alkylating step c) and deprotection step d) are performed successively.

Embodiment 7 is the process according to embodiments 4, 5, or 6 wherein the one or more compounds of formula (V) is 2-methoxymethyl-1,4-benzenediamine (V-a) and are prepared using the steps of:
a) protecting amine functions in one or more compounds of formula (I-a) in the presence of phthalic anhydride in dioxane to prepare one or more compounds of formula (II-a):

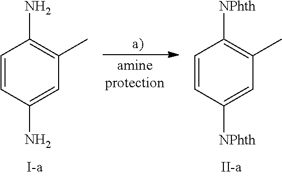

b) performing a radical bromination step of the one or more compounds of formula (II-a) in the presence of a brominating reagent, a radical initiator, and one or more solvents to prepare one or more compounds of formula (III-a):

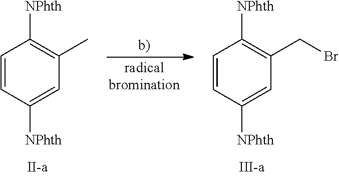

c) performing a methoxylation step by reacting the one or more compounds of formula (III-a) in the presence of a methoxylating reagent and one or more solvents to prepare one or more compounds of formula (IV-a):

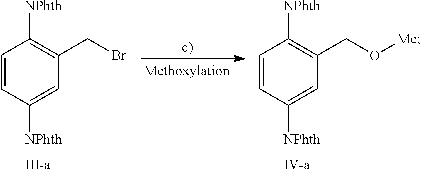

and
d) performing a deprotection step by reacting the one or more compounds of formula (IV-a) in the presence of hydrazine and one or more solvents to prepare the 2-methoxymethyl-1,4-benzenediamine (V-a):

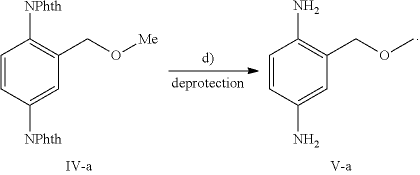

Embodiment 8 is the process according to embodiments 4, 5, 6, or 7 wherein the one or more compounds of formula (V) is 2-methoxymethyl-1,4-benzenediamine (V-a) and are prepared using the steps of:
a) protecting the amine function in the compound of the formula (I-b) in the presence of succinic anhydride in dioxane to prepare the compound of the formula (II-b):

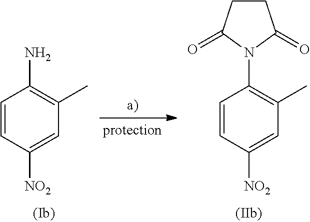 [SMILES: Cc1cc([N+](=O)[O-])ccc1N ; Cc1cc([N+](=O)[O-])ccc1N1C(=O)CCC1=O]

b) performing a radical bromination step b) of the compound of formula (II-b) in the presence of a brominating reagent, a radical initiator, and one or more solvents to prepare the compound of formula (III-b):

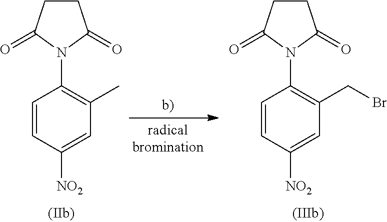 [SMILES: Cc1cc([N+](=O)[O-])ccc1N1C(=O)CCC1=O ; O=C1CCC(=O)N1c1ccc([N+](=O)[O-])cc1CBr]

c) performing a methoxylation step c) by reacting the compound of formula (III-b) in the presence of a methoxylating reagent and one or more solvents to prepare the compound of formula (IV-b):

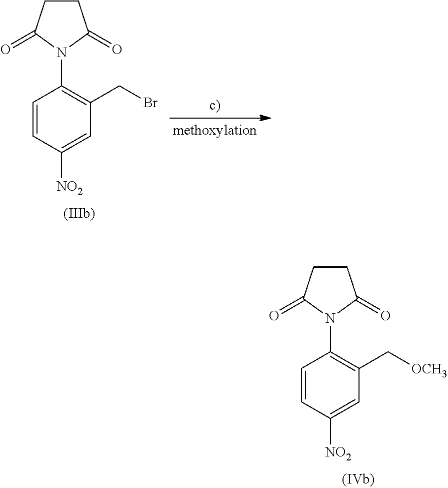 [SMILES: O=C1CCC(=O)N1c1ccc([N+](=O)[O-])cc1CBr ; COCc1cc([N+](=O)[O-])ccc1N1C(=O)CCC1=O]

d) performing a deprotection step d) by reacting the compound of formula (IV-b) in the presence of hydrazine and one or more solvents to prepare the compound of the formula (V-b):

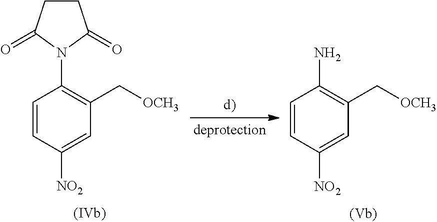 [SMILES: COCc1cc([N+](=O)[O-])ccc1N1C(=O)CCC1=O ; COCc1cc([N+](=O)[O-])ccc1N]

e) performing a reduction step by reacting the compound of formula (V-b) in the presence of hydrogen, a catalyst, and one or more solvents to prepare the 2-methoxymethyl-1,4-benzenediamine (V-a):

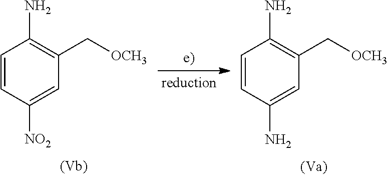 [SMILES: COCc1cc([N+](=O)[O-])ccc1N ; COCc1cc(N)ccc1N]

Embodiment 9 is the process according to embodiments 4, 5, 6, 7, or 8 wherein the one or more compounds of formula (V) is 2-methoxymethyl-1,4-benzenediamine (V-a) and are prepared using the steps of:
a) protecting the amine functions in the compound of the formula (I-c) in the presence of succinic anhydride in dioxane to prepare the compound of the formula (II-c):

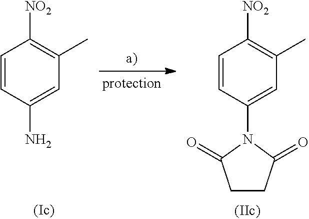 [SMILES: Cc1cc(N)ccc1[N+](=O)[O-] ; Cc1cc(N2C(=O)CCC2=O)ccc1[N+](=O)[O-]]

b) performing a radical bromination step b) of the compound of formula (II-c) in the presence of a brominating reagent, a radical initiator, and one or more solvents to prepare the compound of formula (III-c):

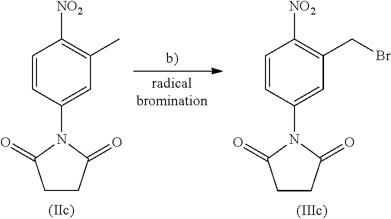 [SMILES: Cc1cc(N2C(=O)CCC2=O)ccc1[N+](=O)[O-] ; O=C1CCC(=O)N1c1ccc([N+](=O)[O-])c(CBr)c1]

c) performing a methoxylation step c) by reacting the compound of formula (III-c) in the presence of a methoxylating reagent and one or more solvents to prepare the compound of formula (IV-c):

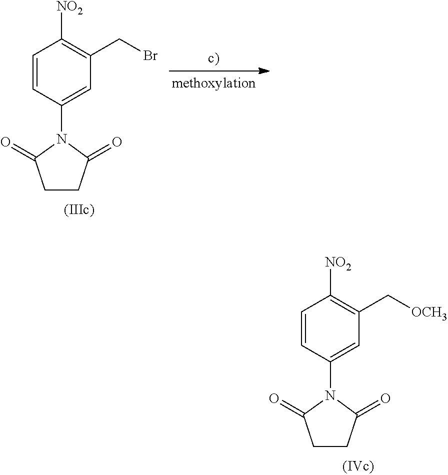 [SMILES: O=C1CCC(=O)N1c1ccc([N+](=O)[O-])c(CBr)c1 ; COCc1cc(N2C(=O)CCC2=O)ccc1[N+](=O)[O-]]

d) performing a deprotection step d) by reacting the compound of formula (IV-c) in the presence of hydrazine and one or more solvents to prepare the compound of the formula (V-c):

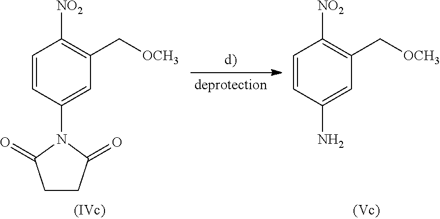 [SMILES: COCc1cc(N2C(=O)CCC2=O)ccc1[N+](=O)[O-] ; COCc1cc(N)ccc1[N+](=O)[O-]]

e) performing a reduction step by reacting the compound of formula (V-c) in the presence of hydrogen, a catalyst, and one or more solvents to prepare the 2-methoxymethyl-1,4-benzenediamine (V-a):

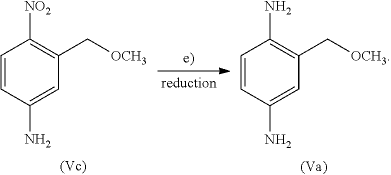 [SMILES: COCc1cc(N)ccc1[N+](=O)[O-] ; COCc1cc(N)ccc1N]

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for preparing a primary intermediate for oxidative dyeing compositions, the process comprising performing a radical halogenation of one or more compounds of formula (II) in the presence of a halogenating agent, one or more radical initiators, heat and one or more solvents to prepare one or more compounds of formula (III): 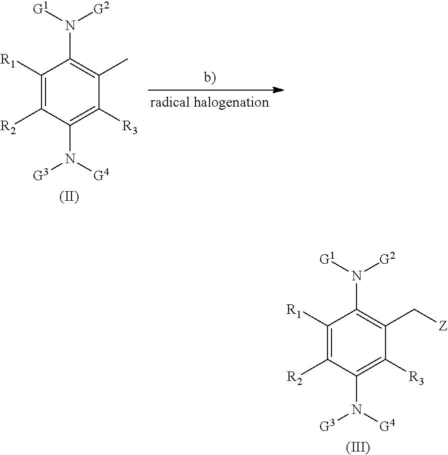
wherein R¹, R², and R³ are substituents independently selected from the group consisting of
(a) C-linked substituents selected from the group consisting of:
(i) aliphatic or heteroaliphatic substituents selected from the group consisting of mono- or poly-substituted or unsubstituted, straight or branched, cyclic or acyclic, saturated or unsaturated, and combinations thereof;
(ii) aryl substituents selected from the group consisting of mono- or poly-substituted or unsubstituted, saturated or unsaturated, and combinations thereof;
(iii) heteroaryl substituents selected from the group consisting of mono- or poly-substituted or unsubstituted, saturated or unsaturated, and combinations thereof;
wherein said substituents of (i), (ii) and (iii) comprise from about 1 to 10 carbon atoms and from about 0 to 5 heteroatoms selected from the group consisting of O, F, N, P and Si;
(b) S-linked substituents selected from the group consisting of SA¹, SO₂A¹, SO₃A¹, SSA¹, SOA¹, SO₂NA¹A², SNA¹A², and SONA¹A²;
(c) O-linked substituents selected from the group consisting of OA¹ and ONA¹A²;
(d) N-linked substituents selected from the group consisting of NA¹A²; (NA¹A²A³)⁺, NA¹SA², NO₂, and NA¹A²;
(e) substituents selected from the group consisting of COOA¹, CONA¹, CONA¹COA², C(═NA¹)NA¹A², CN, and X;
(f) fluoroalkyl substituents selected from the group consisting of mono-, poly-, and per-fluoro alkyl systems comprising from 1 to 12 carbon atoms and from 0 to 4 heteroatoms; and
(g) H;
and combinations thereof;
wherein A¹, A², and A³ are substituents independently selected from the group consisting of H, substituted or unsubstituted C1-C6 alkyl, substituted or unsubstituted C1-C6 heteroalkyl, substituted or unsubstituted aryl, substituted or unsubstituted C1-C6 heteroalkylaryl, substituted or unsubstituted heteroaryl, and/or substituted or unsubstituted cyclic heteroalkyl;
wherein G¹, G2, G³ and G⁴ are substituents selected from the group consisting of hydrogen, oxygen, carbamates, amides, sulfonamides, cyclic imides, and combinations thereof;
wherein G¹, G², G³ and G⁴ are not all oxygen; and
wherein Z is chlorine, bromine, or iodine.

2. The process according to claim 1, further comprising the step of:
protecting amine functions in a compound of formula (I) in the presence of a protecting agent and one or more solvents to prepare the one or more compounds of formula (II) 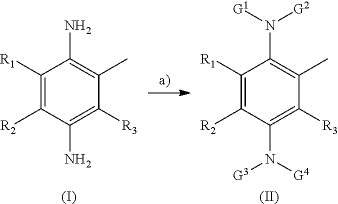
wherein R¹, R², R³ and G¹, G², G³, G⁴ are substituents as defined in claim 1.

3. The process according to claim 1, further comprising the step of:
b) alkoxylating the one or more compounds of formula (III) in the presence of an alkoxylating agent and one or more solvents to prepare the one or more compounds of formula (IV), 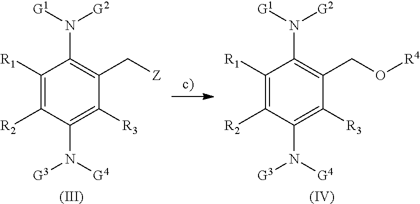
wherein R¹, R², R³, G¹, G², G³, G⁴ and Z are substituents as defined in claim 1; and
wherein R⁴ is a substituent independently selected from H, substituted or unsubstituted C1-C6 alkyl, substituted or unsubstituted C1-C6 heteroalkyl, substituted or unsubstituted aryl, substituted or unsubstituted C1-C6 heteroalkylaryl, substituted or unsubstituted heteroaryl, and/or substituted or unsubstituted heterocyclic aliphatic.

4. The process according to claim 3, further comprising the step of:
a) deprotecting the one or more compounds of formula (IV) in the presence of a deprotecting agent and one or more solvents to prepare one or more compounds of formula (V): 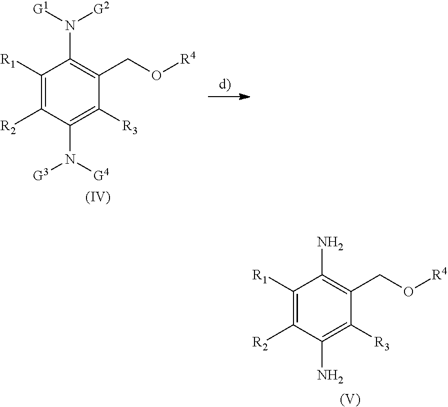
wherein R¹, R², R³, R⁴, G¹, G², G³, and G⁴ are substituents as defined in claim 3.

5. The process according to claim 4, further comprising the step of transformation of the one or more compounds (V) into a salt of one or more compounds of formula (VI) in the presence of an acid mHZ and one more solvents: 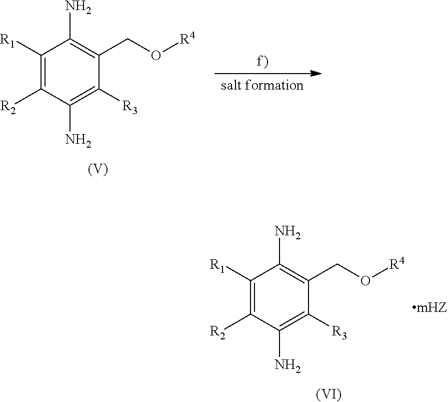
wherein R¹, R², R³ and R⁴ are substituents as defined in claim 4; and
wherein m is 0.5, 1, or 2; and
wherein HZ is selected from the group consisting of D,L-malic, L-malic, D-malic, hydrochloric, hydrobromic, phosphoric, citric, acetic, lactic, succinic, tartaric, sulfuric acids, and mixtures thereof.

6. The process according to claim 4, wherein the amine protection step a), radical halogenation step b), alkylating step c) and deprotection step d) are performed successively.

7. The process according to claim 4, wherein the one or more compounds of formula (V) is 2-methoxymethyl-1,4-benzenediamine (V-a) and are prepared using the steps of:
c) protecting amine functions in one or more compounds of formula (I-a) in the presence of phthalic anhydride in dioxane to prepare one or more compounds of formula (II-a): 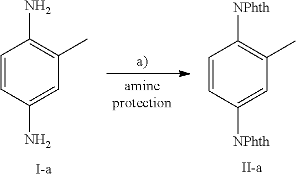
d) performing a radical bromination step of the one or more compounds of formula (II-a) in the presence of a brominating reagent, a radical initiator, and one or more solvents to prepare one or more compounds of formula (III-a): 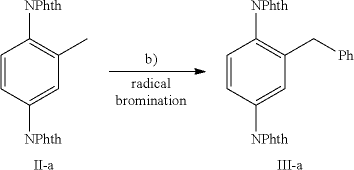
c) performing a methoxylation step by reacting the one or more compounds of formula (III-a) in the presence of a methoxylating reagent and one or more solvents to prepare one or more compounds of formula (IV-a): 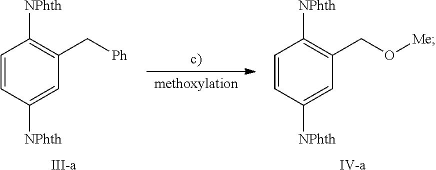
and
d) performing a deprotection step by reacting the one or more compounds of formula (IV-a) in the presence of hydrazine and one or more solvents to prepare the 2-methoxymethyl-1,4-benzenediamine (V-a):

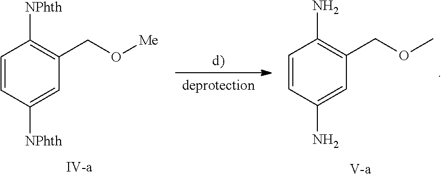

8. The process according to claim 4, wherein the one or more compounds of formula (V) is 2-methoxymethyl-1,4-benzenediamine (V-a) and are prepared using the steps of:
c) protecting the amine function in the compound of the formula (I-b) in the presence of succinic anhydride in dioxane to prepare the compound of the formula (II-b): 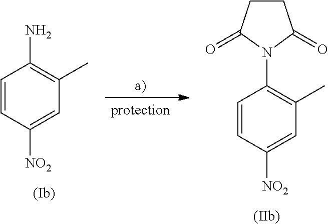
d) performing a radical bromination step b) of the compound of formula (II-b) in the presence of a brominating reagent, a radical initiator, and one or more solvents to prepare the compound of formula (III-b): 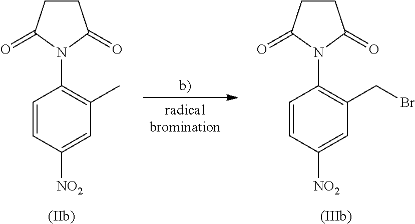
c) performing a methoxylation step c) by reacting the compound of formula (III-b) in the presence of a methoxylating reagent and one or more solvents to prepare the compound of formula (IV-b): 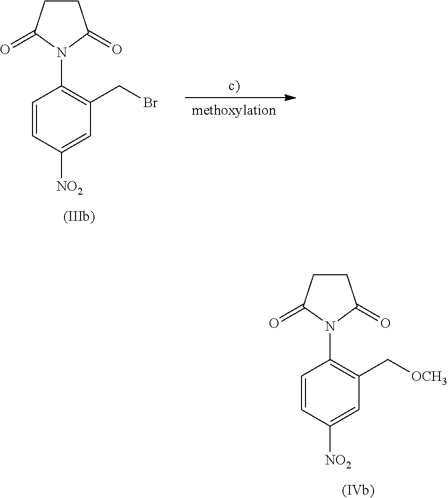
d) performing a deprotection step d) by reacting the compound of formula (IV-b) in the presence of hydrazine and one or more solvents to prepare the compound of the formula (V-b): 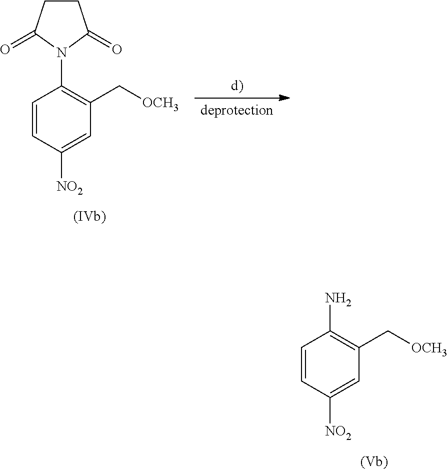
e) performing a reduction step by reacting the compound of formula (V-b) in the presence of hydrogen, a catalyst, and one or more solvents to prepare the 2-methoxymethyl-1,4-benzenediamine (V-a):

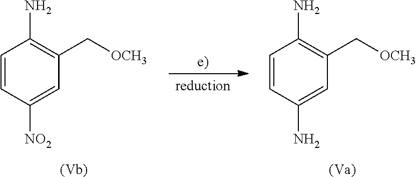

9. The process according to claim 4, wherein the one or more compounds of formula (V) is 2-methoxymethyl-1,4-benzenediamine (V-a) and are prepared using the steps of:
c) protecting the amine functions in the compound of the formula (I-c) in the presence of succinic anhydride in dioxane to prepare the compound of the formula (II-c): 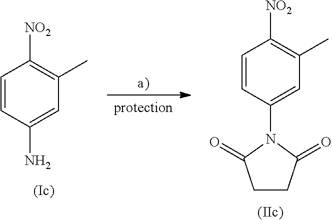
d) performing a radical bromination step b) of the compound of formula (II-c) in the presence of a brominating reagent, a radical initiator, and one or more solvents to prepare the compound of formula (III-c): 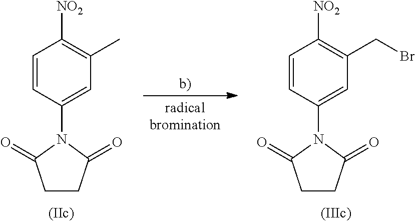
c) performing a methoxylation step c) by reacting the compound of formula (III-c) in the presence of a methoxylating reagent and one or more solvents to prepare the compound of formula (IV-c): 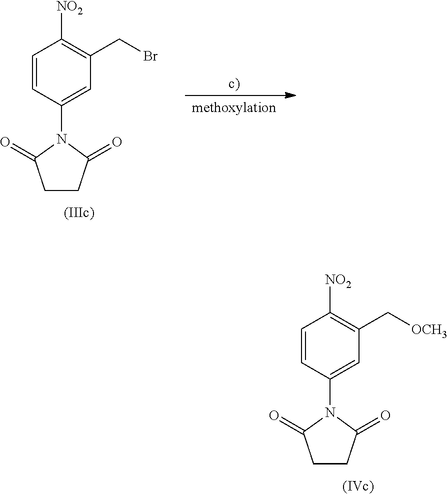
d) performing a deprotection step d) by reacting the compound of formula (IV-c) in the presence of hydrazine and one or more solvents to prepare the compound of the formula (V-c): 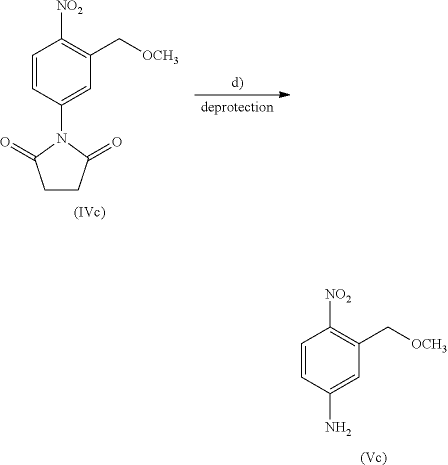
e) performing a reduction step by reacting the compound of formula (V-c) in the presence of hydrogen, a catalyst, and one or more solvents to prepare the 2-methoxymethyl-1,4-benzenediamine (V-a): [chemical structure]
